(19) United States
(12) Patent Application Publication
(10) Pub. No.: US 2011/0130658 A1
(43) Pub. Date: Jun. 2, 2011

(54) ULTRASONIC CAPSULE

(75) Inventor: Gavriel J. Iddan, Haifa (IL)

(73) Assignee: RDC Ltd., Tel-Aviv (IL)

(21) Appl. No.: 12/673,719

(22) PCT Filed: Aug. 14, 2008
(86) PCT No.: PCT/IL08/01124

§ 371 Date: Feb. 10, 2011

Related U.S. Application Data
(60) Provisional application No. 60/935,516, filed on Aug. 16, 2007.

(51) Int. Cl. A61B 8/00

(52) U.S. Cl. 600/437

(57) ABSTRACT

A system and method are described for the purpose of performing non invasive examination of the colon. The proposed method is based on an ingestible capsule incorporating ultrasonic transducer elements, control circuit and an external wireless power supply and data recorder. The recorded ultrasonic data is later processed and a virtual image of the lumen is generated for diagnostic purpose. The proposed method will not require prep and thus will be more patient friendly.

### FIELD OF THE INVENTION

The present invention relates to an apparatus and method for the purpose of performing non invasive examination of the gastrointestinal (GI) tract and more specifically the colon.

### BACKGROUND OF THE INVENTION

Devices and methods for performing in-vivo imaging of passages or cavities within a body are known in the art. Such devices may include, inter alia, various endoscopic imaging systems and devices for performing imaging in various internal body cavities.

Typical current in-vivo imaging devices use light or other electromagnetic energy to form images. Images based on light or other electromagnetic energy may not provide information on, for example, features or structures obscured by the contents of the gastrointestinal (GI) tract or beyond or behind the surface of the lumen being imaged. A medical practitioner may desire to image such structures or features.

Further, when imaging the GI tract, a thorough cleaning may be required beforehand. In particular, the colon may be filled with matter such feces, while other parts of the GI tract may be filled with liquid which is more transparent. However, various parts of the GI tract may also be filled with more opaque matter. Uncomfortable cleaning may for example require a multi day liquid diet or low residue diet, or the use of special cleaning agents such as laxatives.

The use of ultrasonic capsule for the purpose of colon diagnosis was proposed in the past for example US application 20030195415; titled “Device, system and method for acoustic in-vivo measuring”; to Iddan described a capsule incorporating a ring or rings of miniature ultrasonic elements performing an electronic circular scan as it moves through the colon. The gathered data is transmitted to an external recorder for offline processing.

The use of miniature ultrasonic devices for lumen wall imaging is very common in cardiology under the acronym IVUS. The IVUS is a tethered device hence can not be directly adopted for the colon.

Cabled ultrasonic devices are also common for rectal and vaginal examinations thus ultrasound has been proved to be effective imaging modality.

It was observed that one of the major reasons for not adopting the ultrasonic capsule for colon screening is the power problem. The batteries that were successfully used within video capsule will not be able to guarantee the power required by a high density ultrasonic array. The residence time of a capsule inside the colon may be as long as two days or more if accelerating medication is not to be used.

It will therefore beneficial to devise a new approach to the colon screening by a capsule that will not require preparation One should keep in mind that battery operation is a backup if a motion sensing capabilities are incorporated or if a high capacity battery is available.

U.S. Pat. No. 5,604,531; to Iddan, et al.; titled “In vivo video camera system”; discloses an in vivo video camera system and an autonomous video endoscope are described. Each system includes a swallowable capsule, an RF transmitter and a reception system. The swallowable capsule includes a camera system and an optical system for imaging an area of interest onto the camera system. The transmitter transmits the video output of the camera system and the reception system receives the transmitted video output.

U.S. Pat. No. 5,320,104; titled “Transesophageal ultrasound probe”; to Fearnside, James, et. Al.; discloses an endoscopic ultrasound probe with take up cable mechanism which and having a rotatable transducer array with take up mechanism which has flexible cable and as array rotates, cable length alters.

US patent application 2006/0009681 titled “Ultrasonic Endoscope” to T. Tanaka et. Al. describes an ultrasonic endoscope incorporating a circular array much like US application 2003/0195415

### SUMMARY OF THE INVENTION

The present invention relates to an apparatus and method for the purpose of performing non invasive examination of the colon.

According to exemplary embodiment of the current invention a system for non-invasive ultrasonic examination of the GI tract and more specifically the colon is provided. The system comprises a capsule configured and sized to be ingested by or inserted by a tool such as an endoscope fitted with a special capsule holder in human or animal patient and pass through its GI tract.

The said capsule is an ingestible capsule having dome shape ends made of a bio compatible polymer shell and incorporates plurality of ultrasonic (US) elements capable of emitting ultrasonic waves out of the said capsule and detecting ultrasonic waves arriving towards the capsule. The capsule includes in addition:

Drive circuits activating the said elements and receiving circuits for detecting the arriving signals.

Receive/transmit RF circuits for transmitting signals out of the capsule and receiving arriving signals. The capsule preferably further comprises a processing circuit and its imbedded software capable of controlling the drivers, the receivers, the RF section and the power section.

The capsule preferably further comprises power section, incorporating energy storage (battery or capacitor), and energy converter, capable of converting ultrasonic or RF energy to the energy consumed by the capsule.

The capsule optionally further comprises an optical sensor and illumination incorporated in the capsule capable of imaging tissue such as disclosed in U.S. Pat. No. 5,604,531 and its successors.

Said ultrasonic (ULS) capsule generates a probing pulse or pulse train in a plane preferably perpendicular to the capsule axis. The reflected ULS energy creates slice images converted to RF and transmitted out of the body to be picked up by antenna array mounted on the patient body (see FIG. 5). The accumulated slices are later converted in a workstation to a reconstructed colon lumen image. The detected RF and ULS signals are used in addition for capsule localization and orientation this later information helps in the proper lumen image reconstruction.

Although the capsule may have its own power source such as a battery pack, additional power is preferably transferred to the capsule from an out of the body special device via an RF or ULS link.

The RF Energy Transmitter is located out of body on the belt or in the west and delivers the power signal to the antenna array which by proper phasing and optional switching, as is known in the art of phased arrays, delivers the focused RF energy directly to the capsule with maximal efficiency.

Inside the capsule, an antenna attached to tank circuit tuned to the RF frequency; captures the RF energy and delivers it sequentially to each of the Piezoelement at its turn as it forms part of the tank circuit which is equivalent to a LC circuit (comprising an inductor, represented by the letter L, and a capacitor), tuned to be at resonance at least at one preferred frequency. It should be realized that in these embodiments, the electromechanical properties of the piezoelectric elements and/or properties of delay lines may take part in tuning the tank circuit.

According to an aspect of a current invention, an ingestible capsule for non-invasive ultrasound imaging is provided comprising: power receiving antenna capable of receiving RF power; plurality of piezoelectric elements generating ultrasonic signal in response to RF electric signal; beam forming circuit capable of controlling phases of RF signal transferred from said power receiving antenna to said plurality of piezoelectric elements; and a controller controlling said beam forming circuit.

In an exemplary embodiment the frequency of RF power received by said power receiving antenna is the same as frequency of said generated ultrasonic signal.

In an exemplary embodiment the power receiving antenna is part of a tuned circuit tuned to frequency of said received RF power.

In an exemplary embodiment the beam forming circuit comprises at least one delay line.

In an exemplary embodiment the beam forming circuit comprises at least two delay lines.

In an exemplary embodiment the beam forming circuit further comprises switches for controlling the delay of said RF signal delivered to plurality of said piezoelectric elements.

In an exemplary embodiment the plurality of piezoelectric elements converts the reflection from a tissue of a portion of said ultrasonic signal to electric signal indicative of said tissue.

According to an aspect of a current invention, a system for non-invasive ultrasound imaging is provided comprising: ingestible capsule comprising: receiving antenna capable of receiving RF power; plurality of piezoelectric elements generating ultrasonic signal in response to RF electric signal; beam forming circuit capable of controlling phases of RF signal transferred from said power receiving antenna to said plurality of piezoelectric elements; and a controller controlling said beam forming circuit; and external unit comprising: a power transmitting antenna capable of transmitting said RF power to said receiving antenna.

In an exemplary embodiment the plurality of piezoelectric elements converts the reflection from a tissue of a portion of said ultrasonic signal to electric signal indicative of said tissue.

In an exemplary embodiment the electric signal indicative of said tissue is transmitted from said capsule to said external unit.

In an exemplary embodiment the receiving antenna capable of transmitting said signal indicative of said tissue from said capsule to said external unit.

In an exemplary embodiment the capsule further comprising a data transmitter capable of transmitting said signal indicative of said tissue from said capsule to said external unit.

In an exemplary embodiment the external unit comprises sensors capable of sensing location and In an exemplary embodiment the sensors capable of sensing location and orientation of said capsule are ultrasonic sensors.

In an exemplary embodiment the sensors capable of sensing location and orientation of said capsule are RF sensors.

In an exemplary embodiment the system further comprising: a viewing module comprising:

a processor capable of receiving data from said external unit and processing said data to render 3D images based on said data; and a display for displaying said rendered 3D images.

According to an aspect of a current invention, a method of diagnosing the human body is provided comprising the steps of: ingesting an ultrasonic capsule; transmitting RF power from an external unit to said capsule; converting said transmitted RF power to ultrasonic beam emitted by said capsule; reflecting a portion of said ultrasonic beam from human tissue; converting reflected portion of said ultrasonic beam to electronic signal; and transmitting RF signal indicative of said converted electric signal from said capsule to said external unit.

In an exemplary embodiment the step of transmitting RF signal indicative of said converted electric signal comprises transmitting signal at frequency equal to frequency of said RF power.

In an exemplary embodiment the step of transmitting RF signal indicative of said converted electric signal comprises transmitting signal at frequency not equal to frequency of said RF power.

In an exemplary embodiment the method further comprises detecting position and orientation of said capsule in respect to said external unit.

In an exemplary embodiment the method further comprises calculating a 3D image of boundaries of a body lumen through which said capsule is traversing based on signal indicative of said converted electric signal and said detected position and orientation of said capsule in respect to said external unit.

In an exemplary embodiment the method further comprises displaying said calculated 3D image.

In an exemplary embodiment the body lumen through which said capsule is traversing comprises at least a part of a patient's gastrointestinal tract.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 schematically depicts a system for non-invasive ultrasonic examination of the GI tract and more specifically the colon according to exemplary embodiment of the current invention.

FIG. 2 schematically depicts a capsule for non-invasive examination of the GI tract according to exemplary embodiment of the current invention.

FIG. 3a schematically depicts some details of capsule and the corresponding external unit according to a first exemplary embodiment of the current invention.

FIG. 3b schematically depicts some details of capsule and the corresponding external unit according to a second exemplary embodiment of the current invention.

FIG. 4a schematically depicts details of beam forming according to an exemplary embodiment of the current invention.

FIG. 4b schematically depicts details of beam forming according to another exemplary embodiment of the current invention.

FIG. 5 schematically shows an exemplary setup according to an exemplary embodiment of the current invention.

FIG. 6a schematically depicts the ultrasonic imaging capsule and one typically acquired ultrasound slice according to an exemplary embodiment of the invention.

FIG. 6b schematically depicts the reconstruction of 3D image from acquired ultrasound slices according to an exemplary embodiment of the invention.

FIG. 7 schematically depicts the workflow of a typical data processing and evaluation according to an exemplary embodiment of the current invention.

FIG. 8 schematically depicts some images generated during a typical data processing and evaluation according to an exemplary embodiment of the current invention.

FIG. 9 schematically depicts the physician viewing station and some of its display according to an exemplary embodiment of the current invention.

### DESCRIPTION OF THE PREFERRED EMBODIMENTS

The present invention relates to an apparatus and method for the purpose of performing non invasive examination of in vivo body cavities, for example the GI tract and more specifically the colon.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

In discussion of the various figures described herein below, like numbers refer to like parts.

The drawings are generally not to scale.

For clarity, non-essential elements and parts obvious to professionals were omitted from some of the drawings.

As used herein, an element or step recited in the singular and proceeded with the word “a” or “an” should be understood as not excluding plural elements or steps, unless such exclusion is explicitly recited.

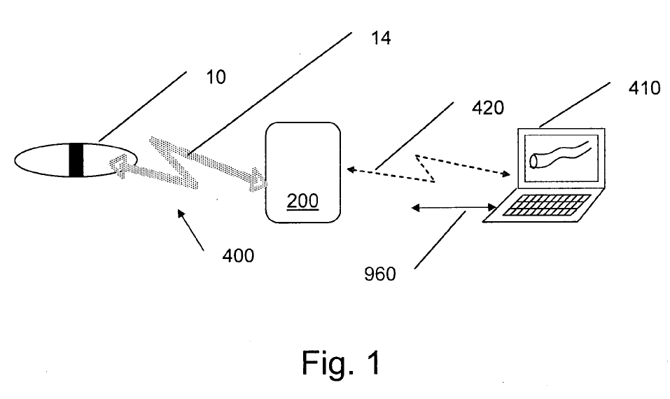

FIG. 1 schematically depicts a system for non-invasive ultrasonic examination of the GI tract and more specifically the colon according to exemplary embodiment of the current invention.

System 400 comprises a capsule 10, configured and sized to be ingested by a human or animal patient and pass through its GI tract.

Ultrasonic arrays are commonly used for imaging of body lumens, including the GI tract, blood vessels, vagina and the urinary vessels. Endoscope with a scanning sensor and array sensors are in use for many years hence the art of designing and fabricating a circular or rectangular sensor array is considered common knowledge. Existing devices known in the art draw their power from the outside via a cable which naturally is unacceptable for a capsule. The attempt to use batteries in a capsule for the operation of the ultrasonic array will limit the duration of operation hence a battery may be considered as a limited duration power backup. It is therefore advantageous to deliver the power required for the ultrasonic beam from the outside via a wireless method and thus enabling practically unlimited operation of the capsule.

Capsule 10 is in RF communication 14 with external unit 200 positioned in proximity to and preferably in contact to the patient body. External unit 200 comprises an RF transmitter/receiver and a data recorder. External unit 200 may comprises some data processing capability as well as mobile power source such as a battery to allow the patient to move about while the imaging take place.

External unit 200 may establish a link 420 to a data processing and viewing station 410. Viewing station 410 may be a computer such as a PC, a laptop, a work station or a dedicated computing and viewing unit.

Link 420 may be a wireless link such as RF link (e.g. Bluetooth, Wi-Fi, cellular, etc.), alternatively or additionally a cable (e.g. LAN or USB cable, etc.) may be used; alternatively or additionally data may be stored at external unit 200, for example on a memory card or a removable data storage device such as disk-on-key and retrieve (e.g. data card, disk on key) and physically removed from external unit 200 and be inserted to Viewing station 410.

Optionally, viewing station 410 is connected through optional communication link 960 such as LAN, phone modem, cellular modem, etc. to remote a sites such as central servers and optionally to hospital Picture Archiving and Communication System (PACS) and/or hospital billing system.

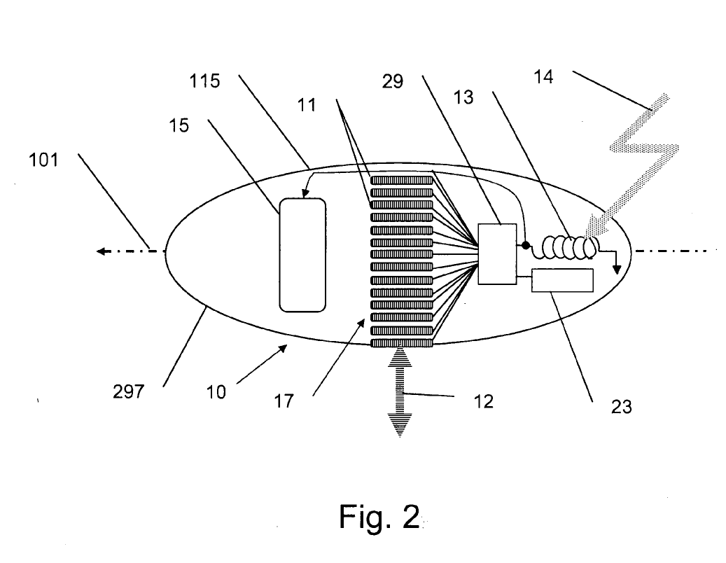

FIG. 2 schematically depicts a capsule for non-invasive examination of the GI tract according to exemplary embodiment of the current invention.

Capsule 10 is encapsulated in bio-compatible shall 297 sized to be ingested by a patient or inserted. Preferably, the capsule is elongated and has a long axis 101. When moving in the patient's GI tract, capsule 10 may advance such as long axis 101 is pointing at or approximately at the direction of motion and thus long axis 101 is approximately parallel to the axis of the GI cavity in which capsule 10 is advancing. Alternatively, other shapes of a capsule may be used, for example a sphere or a pill having three different axis lengths or a round pill having one short axis.

In an exemplary embodiment of the invention, capsule 10 comprises a circular circumferential piezoelectric transducer array; wherein elements of piezoelectric transducer array may comprise crystals, ceramics, polymers and MEMS transuding elements. Piezoelectric transducer array 17 comprises a plurality of piezoelectric elements 11. The ultrasonic transducer elements can be arranged in a variety of forms such as rectangular, strip and a circular array or a plurality of arrays which will be addressed in more details. Ultrasonic transducer array 17 is used as phased array, forming an ultrasonic imaging beam 12. Optionally, transducer array 17 is of a shape other than circular. Optionally, transducer array 17 is a 2D array capable of forming and scanning beam 12. Optionally, two or more transducer arrays are used. For example, two arrays may be used, situated at opposite sides of the capsule's center an angled in respect to long axis 101 such that each array is capable of imaging objects in a preferably wide conical scanning zone each angled to, and surrounding opposite sides of long axis 101. Optionally three transducer arrays are used, for example one equatorial and two preferably longitudinally situated such that they are perpendicular to each other at the poles.

RF link 14 transmits RF power from external unit 200 to capsule 10. RF power is received by Receiver RF antenna 13. Antenna 13 is preferably a tuned coil, tuned to the RF frequency, however other types of antennas may be used, for example whip antenna extending outside pill 10, printed antenna formed on a PCB or on the shell of the pill, etc.

It is an aspect of the current invention to deliver the energy required for the operation of the ultrasonic elements via a RF link.

Preferably, the RF power delivered to the capsule is already at the frequency of the ultrasonic signal used for ultrasound imaging. Penetration depth and resolution of ultrasound imaging depends on the frequency used, wherein lower frequency is used for deep tissue imaging ad high frequency is used for high resolution shallow imaging. Axial resolution achieved using short ultrasonic pulses to avoid ambiguity. Generation of short pulses necessitates high frequency and large bandwidth. Transverse (angular) resolution necessitates imaging system of large F number, which necessitates the use of wide transducer array and short ultrasonic wavelength (that is—high frequency).

Preferably, high frequency is used according to the current invention to achieve high radial and tangential resolution given the small dimension of the pill. Additionally, shallow imaging is possible as the main information required is imaging of the body cavity boundary which is optionally close to the pill and may appear as large ultrasonic reflection due to the large difference in mechanical properties between the cavity content and the cavity wall.

In some embodiments, antenna 13 is preferably a tuned RF resonance coil, tuned to the RF frequency used. In some embodiment, coil 13 has a large cross section, occupying substantial space in capsule 10 so as to effectively receive RF radiation.

In an exemplary embodiment of the invention, some of the RF energy received by antenna 13 is diverted through power input line 115 to power source 15. Power source 15 supply electrical power to operate electronic circuits within capsule 10. Power source 15 may comprise electrical rectification and power conditioning circuits for generation of DC electrical power from the received RF power. Preferably, a power storage device such as capacitor or rechargeable battery within power source 15 is used for smoothing the rectified signal and to maintain power supply between FR pulses. Optionally, battery within power source 15 is capable to maintain operation of capsule 10 for a duration in the absence of received RF power.

Beam forming electronics 29 controls RF signal delivered to piezoelectric elements 11 of piezoelectric transducer array 17. Beam forming electronics 29 comprises of RF switches and optionally RF phase shifting devices for controlling the RF signal delivered to piezoelectric elements 11. Beam forming controller 23 controls the switches and phase shifters in Beam forming electronics 29 such that ultrasonic beam 12 is properly formed, aimed and optionally focused. Phase shifters in Beam forming electronics 29 may be for example in the form of controlled delay lines or voltage controlled capacitors in RF resonance circuit.

Additionally, capsule 10 may further comprise data receiver and or transmitter, a central controller, data logging device, additional sensors and other structures, some will be detailed in the following figures.

Control signals transmitted by external unit 200 and received by receiver in capsule 10 may be used for turning on or off the ultrasonic beam 12, to alter the scanning pattern or sequence of beam 12, to initiate data transfer from capsule 10 to external unit 200, etc. Additionally, data transferred from capsule 10 to external unit 200 may be raw RF signal as received by elements 11 of array 17, partially processed data, for example time evolution of the ultrasonic signal reflected from the tissue; as well as sensor reading and status indicating signals.

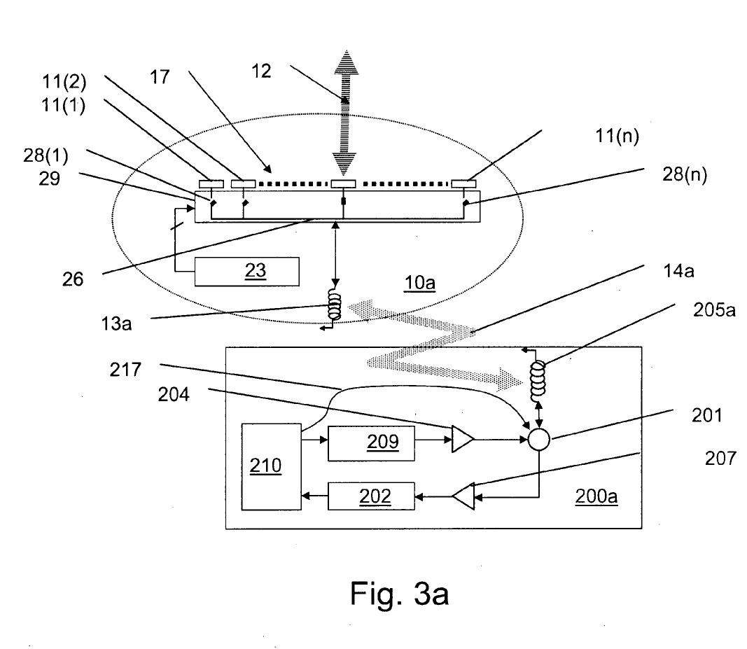

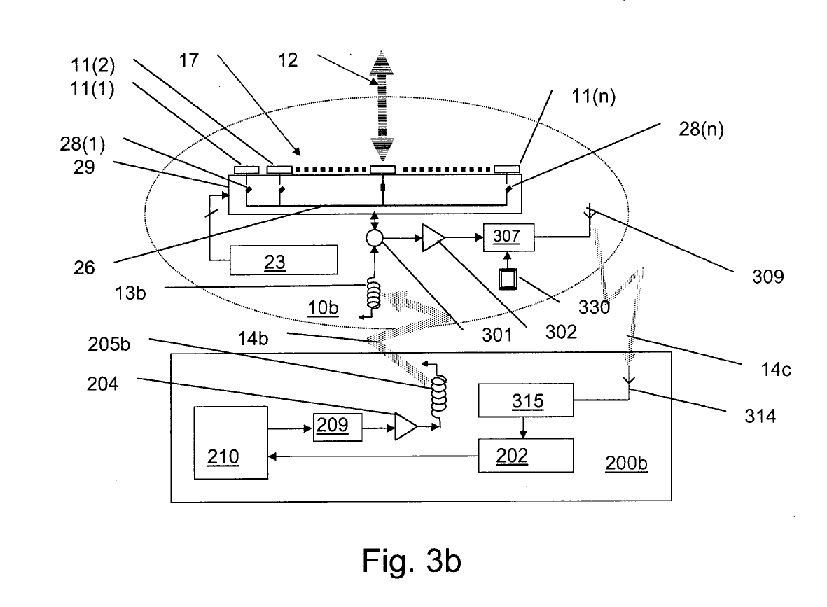

FIG. 3a schematically depicts some details of capsule 10a and the corresponding external unit 200a according to a first exemplary embodiment of the current invention.

For simplicity, some elements seen in other figures or disclosed in the text were omitted from this drawing. As like numbers refer to like elements, discussion of elements already discussed would be omitted.

Capsule 10a is adopted to receive RF power and transmit signal indicative of the ultrasonic signal reflected from the tissue over the same FR link using antenna 13a adopted to both receive RF energy from; and transmit RF data to external unit 200a over two-ways RF link 14a.

In this figure, internal details of beam forming electronics 29 is seen showing the drivers (switches and/or phase shifters) 28(1) to 28(n) driving piezoelectric element 11(1) to 11(n) respectively. The switches and relative phases set by drivers 28 direct ultrasonic beam 12 in a specific direction and optionally focus the beam at a specific distance. Same switches and relative phases set by drivers 28 unite the RF signals from elements 11 creating preferential sensitivity of array 17 to ultrasonic wave reflected from the same specific zone in the tissue to which beam 12 was directed.

According to the first exemplary embodiment of the current invention, the RF signals generated by piezoelectric elements 11, after combined in beam forming electronics 29 is amplified, preprocessed and transmitted by transceiver and antenna 13a to external unit 200a where it is received by antenna 205a. It should be noted that external unit 200a may comprise of plurality of s 205a working together or one or few at a time using multiplexer(s). Additionally, Power transmitting and signal receiving functions of antenna 205a may be split to different antennas such that transmission is done by one antenna and reception by another.

External unit 200a according to a first exemplary embodiment of the current invention comprises an RF generator 209. Optionally, RF generator 209 generates train of RF pulses having pulse duration compatible with the ultrasonic pulse of beam 12. RF power amplifier 204 amplifies the RF pulse. Preferably, RF mixer 201, preferably controlled by control by controller 210 through control line 217 is used to allow transmission of RF power from RF power amplifier 204 to antenna 205a during RF power transmission, and direct RF signal indicative of reflected ultrasonic signal from antenna 205a to signal amplifier 207. Preferably RF mixer 201 is a voltage controlled mixer or switch. RF mixer 201 and/or limiter may also serve to isolate signal amplifier 207 from high power transmitted RF from power amplifier 204 which may damage it.

Amplified signal from signal amplifier 207 is decoded by data decoder 202 and processed data is transferred to controller 210.

Controller 210 preferably comprises data logger for logging processed data.

External unit 200a is externally powered using main power line or powered by a battery pack.

FIG. 3b schematically depicts some details of capsule 10b and the corresponding external unit 200b according to a second exemplary embodiment of the current invention.

For simplicity, some elements seen in other figures or disclosed in the text were omitted from this drawing. As like numbers refer to like elements, discussion of elements already discussed would be omitted.

Capsule 10b is adopted to receive RF power over power FR link 14b using antenna 13b adopted receive RF energy from external unit 200b; and transmit signal indicative of the ultrasonic signal reflected from the tissue over data RF link 14c using data transmitting antenna 309.

In this figure, internal details of beam forming electronics 29 is seen showing the drivers (switches and/or phase shifters) 28(1) to 28(n) driving piezoelectric element 11(1) to 11(n) respectively. The switches and relative phases set by drivers 28 direct ultrasonic beam 12 in a specific direction and optionally focus the beam at a specific distance. Same switches and relative phases set by drivers 28 unite the RF signals from elements 11 creating preferential sensitivity of array 17 to ultrasonic wave reflected from the same specific zone in the tissue to which beam 12 was directed. The RF energy transmitted from 200 is preferably directly converted to ultrasonic energy by having the piezo-element designed as part of the LC tuned tank receiving circuit.

External unit 200b according to a second exemplary embodiment of the current invention comprises an RF generator 209. Optionally, RF generator 209 generates train of RF pulses having pulse duration compatible with the ultrasonic pulse of beam 12. RF power amplifier 204 amplifies the RF pulse which is transmitted to capsule 10b through power transmitting 205b antenna.

RF power is received by power receiving antenna 13b in capsule 10b. Preferably, RF mixer 301 is used to allow transmission of RF power from power receiving antenna 13b to beam forming electronics 29 during RF power transmission, and direct RF signal indicative of reflected ultrasonic signal from beam forming electronics 29 to signal amplifier 302. Preferably RF mixer 301 is a voltage controlled mixer or switch. RF mixer 301 may also serve to isolate signal amplifier 302 from high power transmitted RF from power receiving antenna 13b may damage amplifier 302.

Data transmitter 307 transmits RF signal indicative of reflected ultrasonic signal from capsule 10b to external unit 200b through RF link 14c using data transmitting antenna 309. Optionally, Data transmitter 307 performs additional data conditioning such as one or few of: decoding signal indicative of reflected ultrasonic beam from tissue; converting signal to digital form; signal conditioning and averaging; data reduction and compressing; etc. Optionally, Data transmitter 307 further comprises data storage such as volatile or non-volatile memory for storing data while RF link is not functioning or unavailable. For example, data may be transferred from capsule 10b in bursts, for example using the same power RF frequency while RF power is not transmitted or its transmission is halted. In this case, power receiving antenna 13b may be used as data transmission antenna 309.

It should be noted that external unit 200a may comprise of plurality of elements s 205a working together or one or few at a time using multiplexer(s).

Signal from data RF data link is intercepted by data receiving antenna 314 and converted to electronic signal by data receiver 315. Data is decoded by data decoder 202 and recorded by data recorder in controller 210.

Optionally, additional sensor(s) 303 is housed in capsule 10 (e.g. 10a or 10b). Sensor 303 is used for measuring bio-parameters such as pressure to indicate the arrival into the colon are inter alia incorporated and can be used to trigger the operation of capsule 10 and the external unit 200 (200a or 200b).

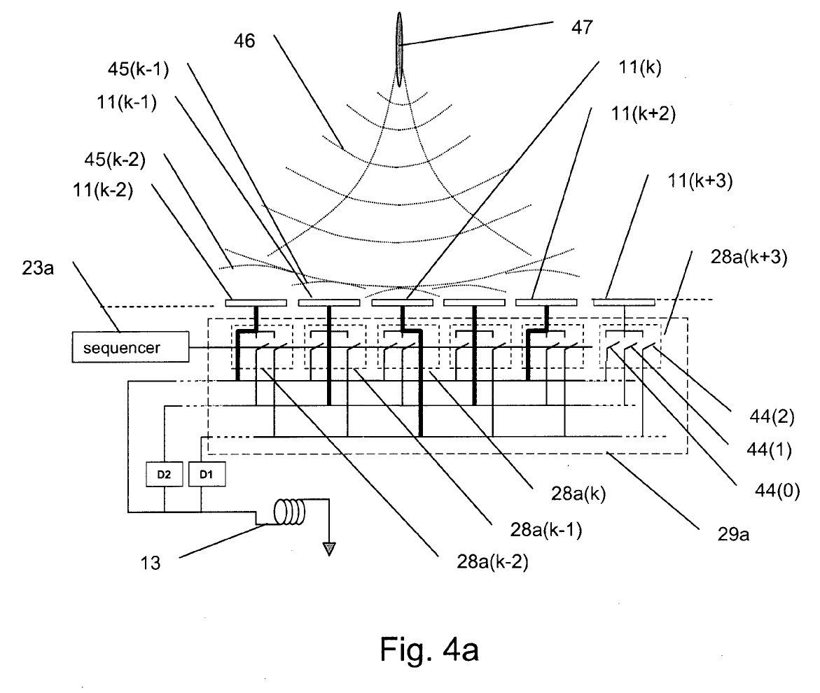

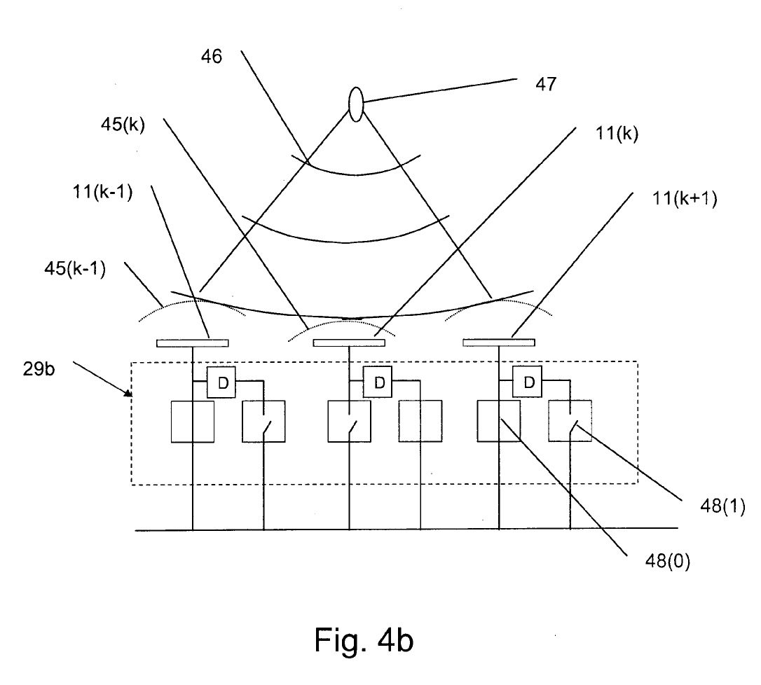

FIG. 4a schematically depicts details of beam forming according to an exemplary embodiment of the current invention.

According to the general aspect of the exemplary embodiment, RF signal received in antenna 13 may arrive at a specific piezoelectric element 11(i) through the corresponding phase controller 28(i) in beam forming electronics 29.

In the embodiment depicted in FIG. 4a, RF signal from antenna 13 is subjected to one of: no delay; time delay of D2 and time delay of D1, wherein time delay of D2 is larger than time delay of D1.

In this embodiment, beam forming controller 23a is a sequencer controlling the switches 44(0); 44(1) and 44(2) within each of phase controller 28a(i) in beam forming electronics 29a. In this example, all three switches are open in phase controller 28a(k+3), thus element 11(k+5) does not receive electrical signal and do not produce ultrasonic pressure wave.

In contrast, switch 44(0) is closed in phase controllers 28a(k−2) and 28a(k+2), thus elements 11(k−2) and 11(k+2) receive un-delayed RF signals. Similarly, switch 44(1) is closed in phase controllers 28a(k−1) and 28a(k+1), thus elements 11(k−1) and 11(k+1) receive RF signals delayed by time delay D2. Similarly, switch 44(2) is closed in phase controllers 28a(k), thus element 11(k) receive RF signal delayed by time delay D1 larger than D2.

Consequently, ultrasonic pressure waterfronts 45 generated by elements 11 are retarded with respect to each other. Interference among ultrasonic pressure waterfronts 45 creates converging ultrasonic pressure waterfront 46 which is focused at ultrasonic focal zone 47.

It should be noted that elements 11 appears as situated on one plane. However, generally elements 11 may be situated on an arc, as is the case of circular array 17 in ultrasonic pill 10. However, by choosing the correct values of D1 and D2, correct focusing may be achieved.

It should be realized that in this embodiment, five elements (k−2 to k+2) are active at each time, and that the beam thus formed is directed radially from the pill's center. By choosing the central element (k), the aiming of the beam is chosen. Imaging of the entire circle around the pill is thus possible.

It should be noted that more (or less) than two delay lines may be used and the number of active element at each point may vary. Additionally, beams aimed at different enough directions, such that the ultrasonic signals are well separated (for example at the two opposite radial directions) may be used. This method is easier to implement using the embodiment of FIG. 3b.

It also should be noted that different aiming (not perpendicular to the array 17) is also possible by using asymmetric delaying of the signals. An advantage of this embodiment is the use of limited number of delay line. It should be noted that a tapped delay line may be used instead of discrete D1 and D2 lines.

FIG. 4b schematically depicts details of beam forming according to another exemplary embodiment of the current invention.

In this embodiment, signal to each element 11 may receive a direct signal via beam forming electronics 29b; through switch 48(0) or a signal delayed by time delay D through switch 48(1), or non at all.

In this exemplary embodiment, three elements (k−1, k, and k+1) are used. It should be noted that more delay lines and switches may be used.

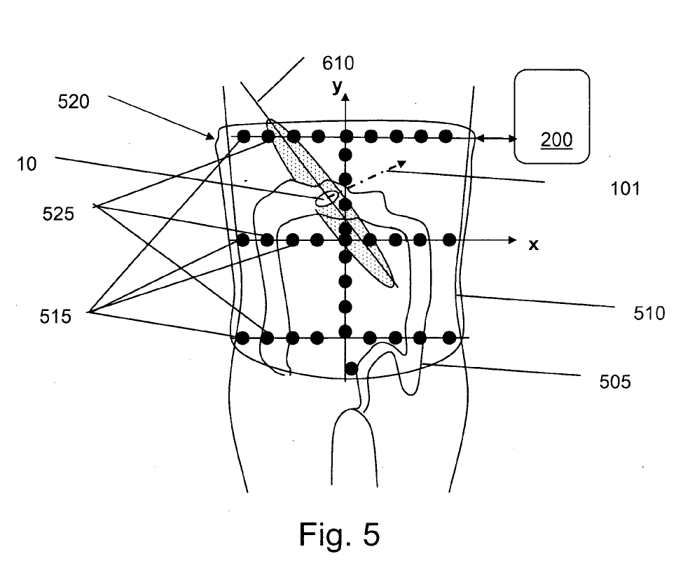

FIG. 5 schematically shows an exemplary setup according to an exemplary embodiment of the current invention.

Preferably a transceiver jacket 520 is attached to patient body 510 or worn by patient incorporating an array of individual RF antenna elements 525 as is known in the art of video capsules. RF antenna elements 525 may be a combination of one or few of antennas 205a, 205b and 314.

According to an exemplary embodiment of the current invention, additional Ultrasound sensors 515 are added to detect the capsule emitted ULS pulses said pulses enable the detection of the capsule positioning and orientation while ultrasonic data is acquired. By knowing the capsule's situation, for example as it traverses the colon 505 of patient 510, each of the ultrasonic slices that are acquired may later be used for the lumen reconstruction. RF power is supplied from external unit 200 to RF antenna elements 525. Ultrasonic data from Ultrasound sensors 515 and RF antenna elements 525 as well as RF data from RF antenna elements 525 is supplied to external unit 200.

Position of the ultrasonic sensors mounted on belt or jacket is known in respect to patient's coordinate system (only x and y are marked in this figure). Signals detected by sensors are used to triangulate the capsule position and orientation thus to identifies the ultrasound imaging plane 610 by locating the capsule position {x, y, z}, the tilt of capsule's long axis 101 and the rotation of the ultrasound acquired slice (not marked in this figure).

According to some embodiments of the invention, the individual sensors are each connected to the recording device via proper cable, RF or IR link such as Bluetooth. At the said recorder the data is optionally muxed and recorded on solid state or magnetic media. A timing clock signal is incorporated in addition to help in the reconstruction.

The recording device may be housed away from the body as a separate unit.

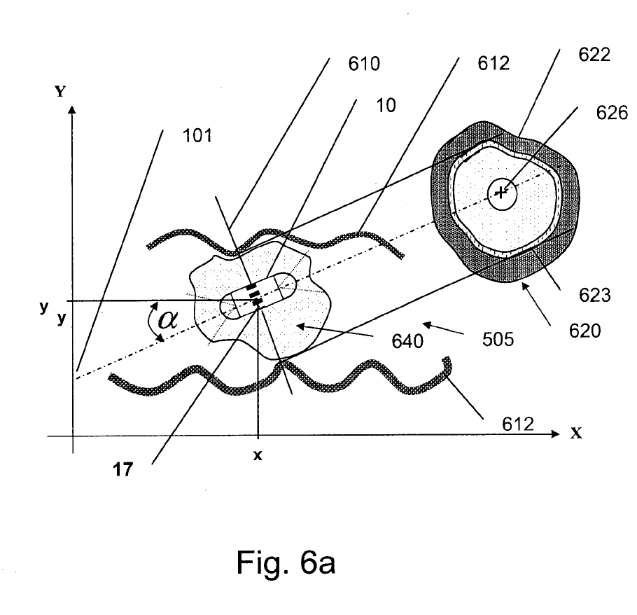

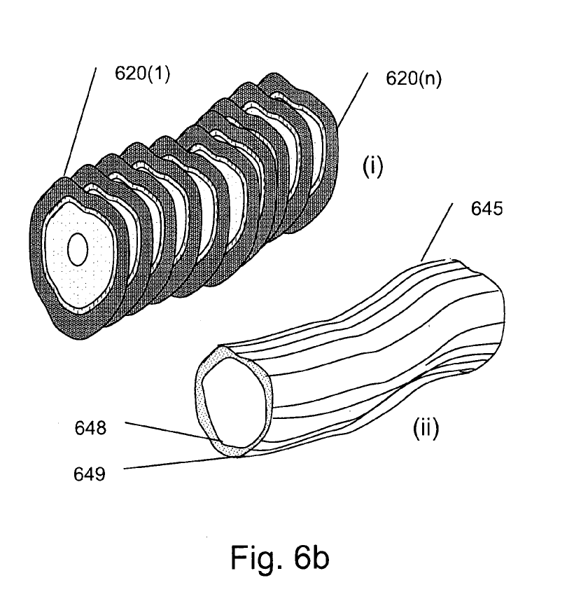

FIG. 6a schematically depicts the ultrasonic imaging capsule and one typically acquired ultrasound slice according to an exemplary embodiment of the invention.

Capsule 10 having an axis 101 is traversing a body cavity such as the colon 510.

Using the (preferably circular) ultrasonic array 17, a 2D ultrasonic image is acquired of the plane 610, defined by the array 17 and preferably perpendicular to capsule axis 101. In this figure, plane 610 is viewed from the side and thus appears as a line. The 2D slice image 620 is centered (626) at body coordinates {x,y,z} (only x and y coordinates are seen in this simplified drawing. It should be noted that plan 610 is generally may be inclined with respect to patient body coordinates, by tilt angles α and β (only α is seen in this simplified drawing) and rotation angle φ about axis 101 (not marked in this simplified drawing).

Plane 610 intersects with the Colon wall 612. In the 2D slice image 620, the Colon wall 612 may be identified (622) by its characteristic ultrasonic properties and ultrasonic reflection and the lumen boundaries 623 identified. By

Lumen contents 640 may have complex appearance and structure depending on its composition. However, using image processing software it is possible to automatically identify the lumen boundaries 623 in image 620 due for example to the contrast between elastic properties of rectal wall 612 and the rectal content 640 which may cause a strong ultrasound reflection from the lumen boundary 623.

Using trigonometry, each image point in image 620, and specifically each image point in colon wall 612 can be matched with a specific coordinate in the 3D patient coordinate system.

As capsule 10 advances and tumbled through the colon or other body cavity such as the intestine, a 3d image of sections or the entire cavity may be formed by data extracted from plurality of 2D images taken at different capsule locations and inclination angles.

FIG. 6b schematically depicts the reconstruction of 3D image from acquired ultrasound slices according to an exemplary embodiment of the invention.

FIG. 6b schematically depicts the reconstructed 3D image according to an exemplary embodiment of the invention.

FIG. 6b(i) depicts plurality of acquired slices 620(1) to 620(n) already correctly positioned and oriented in the patient coordinate system. It should be noted that the preferably, slices 620 are sequentially numbered in chronological order, but slices may be overlapping partially and intersect with each other giving rise to duplication in the data set. In this case, data averaging and other statistical data analysis methods, such as majority rules, may be used to reconstruct the 3D image of the rectal (or other lumen) wall 645 depicted in FIG. 6b(ii).

Optionally, proper localization and orientation of each and every one of the images previously obtained is performed using data obtained from the information gathered by the ultrasound sensors and by data gathered by RF antenna array by known phased array technique.

Once at proper location and orientation, individual images are rendered and a continuous envelope is formed, thus creating a reconstructed lumen having both inner layer 648 and outer layer 649.

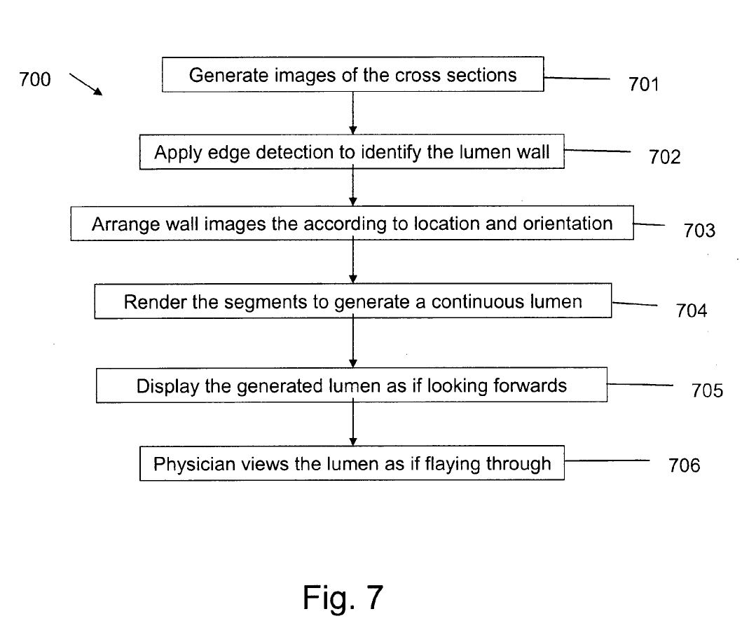

FIG. 7 schematically depicts the workflow of a typical data processing and evaluation according to an exemplary embodiment of the current invention.

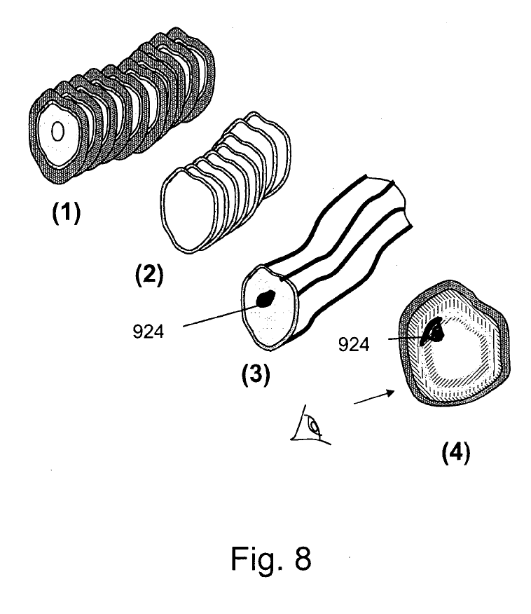

FIG. 8 schematically depicts some images generated during a typical data processing and evaluation according to an exemplary embodiment of the current invention.

Image processing is performed using software executed by viewing station 410. FIG. 7 schematically depicts steps of processing algorithm block flow diagram 700

Slices 620(1) to 620(n) are generated “Generate images of the cross sections” 701; also seen in FIG. 8(1).

Edge detection type algorithm is applied on each of the cross section images 620 to enhance the lumen wall 612 and eliminate the lumen contents 640 in step “Apply edge detection to identify the lumen wall” 702; also seen in FIG. 8(2).

Data from processed slices is registered with patient coordinates at step “Arrange wall images the according to location and orientation” 703.

A 3D image of the rectal (or other lumen) wall 645 is created in step “Render the segments to generate a continuous lumen” 704; also seen in FIG. 8(3). A polyp 924 may be noticed in this image as an inwards protrusion of the lumen wall.

Perspective views of the inner lumen are generated, preferably oriented along the local lumen axis in step “Display the generated lumen as if looking forwards” 705; also seen in FIG. 8(4).

A physician views the perspective views of the inner lumen sequentially in the step “Physician views the lumen as if flaying through” 706; also seen in FIG. 8(5). The physician may identify polyps or other suspected structures and locations such as for example irregularities of the lumen wall. The physician than can use navigation and processing tool to enhance his (her) ability to diagnose the irregularity by performing one or few of: stopping the progress of the “fly through”; pause, move back or forward along the lumen axis; change magnification and image contrast; view raw and differently processed data associated with the locality of the suspected area; change perspective “point of view”; etc.

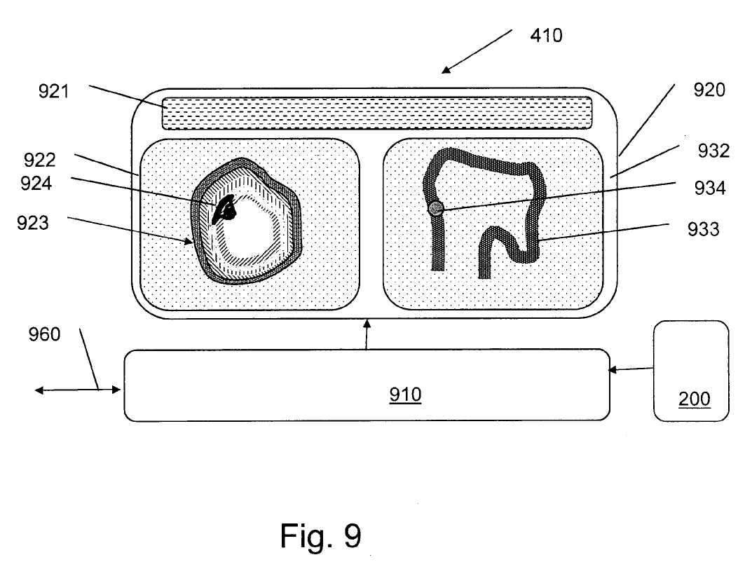

FIG. 9 schematically depicts the physician viewing station and some of its display according to an exemplary embodiment of the current invention.

Viewing station 410 comprises of a data processing unit 910 such as a PC, a workstation, laptop computer, etc. 910. Data processing unit 910 receives image data from external unit 200, processes the image data and displays the processed data preferably together with other information on display 920.

Display 920 may be a dual screen or multi-window display.

According to an exemplary embodiment of the invention the information displayed comprises:

Textual information 921 comprising for example one or few of: patient details; imaging time and date; etc. This section may also comprise of input zones for imputing physician's report and diagnosis.

Display 920 may comprise an image window 922; showing for example: a Single cross section image 923 indicating a polyp 924; or fly-through images of FIG. 8(4).

Display 920 may comprise a navigation and orientation window 932. Navigation and orientation window 932 may be smaller than image window. Navigation and orientation window 932 may be showing for example one or few of: schematic view (from anatomic book or atlas) or actual projection (from 3D rendering) of colon 933; location 934 of present view 923 in window 922; optionally—a vector showing direction of current view; and navigation, zoom and orientation tools.

The reconstructed lumen may be displayed on a monitor side by side with the colon schematic shape, thus, the physician using the system can easily maneuver virtually in and out of the lumen as shown in the figure. If the capsule has in addition a video camera capable of sensing any portion of the spectrum the captured video image may also be presented to the physician.

Optional communication channel 960 (such as LAN, phone line modem, ADSL, internet access, etc.) may be used to connect data processing unit 910 to remote sites such as remote servers; remote data storage devices and optionally to hospital Picture Archiving and Communication System (PACS) and billing.

Indirect mode of operation: The data stored inside the recording device as raw and optionally as slices data may be delivered to the work station following the indication that the capsule has left the patient body or passed the interesting section (E.g. once it reaches the anal cavity where image is not interesting as this zone is subjected to digital examination).

Direct, on line mode of operation: Optionally, viewing does not require the complete colon gathered data. According to this exemplary embodiment, images are presents to the physician in real time. Optionally, instantaneous ultrasound images as well as video images if available are presented.

A combination or real time and indirect operation may be achieved as real time display may be recorded to be replayed later. Viewing may be done at the clinics or hospital or at a remote site such as using a browser at the physician's home or office.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A ingestible capsule for non-invasive ultrasound imaging comprising:
power receiving antenna capable of receiving RF power;
plurality of piezoelectric elements generating ultrasonic signal in response to RF electric signal;
beam forming circuit capable of controlling phases of RF signal transferred from said power receiving antenna to said plurality of piezoelectric elements; and
a controller controlling said beam forming circuit.

2. The capsule of claim 1 wherein frequency of RF power received by said power receiving antenna is the same as frequency of said generated ultrasonic signal.

3. The capsule of claim 1 wherein said power receiving antenna is part of a tuned circuit tuned to frequency of said received RF power.

4. The capsule of claim 1 wherein said beam forming circuit comprises at least one delay line.

5. The capsule of claim 4 wherein said beam forming circuit comprises at least two delay lines.

6. The capsule of claim 4 or 5 wherein said beam forming circuit further comprises switches for controlling the delay of said RF signal delivered to plurality of said piezoelectric elements.

7. The capsule of claim 1 wherein said plurality of piezoelectric elements converts the reflection from a tissue of a portion of said ultrasonic signal to electric signal indicative of said tissue.

8. A system for non-invasive ultrasound imaging comprising:
ingestible capsule comprising:
receiving antenna capable of receiving RF power;
plurality of piezoelectric elements generating ultrasonic signal in response to RF electric signal;
beam forming circuit capable of controlling phases of RF signal transferred from said power receiving antenna to said plurality of piezoelectric elements; and
a controller controlling said beam forming circuit; and external unit comprising:
a power transmitting antenna capable of transmitting said RF power to said receiving antenna.

9. The system of claim 8 wherein said plurality of piezoelectric elements converts the reflection from a tissue of a portion of said ultrasonic signal to electric signal indicative of said tissue.

10. The system of claim 9 wherein said electric signal indicative of said tissue is transmitted from said capsule to said external unit.

11. The system of claim 10 wherein said receiving antenna capable of transmitting said signal indicative of said tissue from said capsule to said external unit.

12. The system of claim 10 wherein said capsule further comprising a data transmitter capable of transmitting said signal indicative of said tissue from said capsule to said external unit.

13. The system of claim 8 wherein said external unit comprises sensors capable of sensing location and orientation of said capsule.

14. The system of claim 13 wherein said sensors capable of sensing location and orientation of said capsule are ultrasonic sensors.

15. The system of claim 13 wherein said sensors capable of sensing location and orientation of said capsule are RF sensors.

16. The system of claim 13 and further comprising:
a viewing module comprising:
a processor capable of receiving data from said external unit and processing said data to render 3D images based on said data; and
a display for displaying said rendered 3D images.

17. A method of diagnosing the human body comprising the steps of:
ingesting an ultrasonic capsule;
transmitting RF power from an external unit to said capsule;
converting said transmitted RF power to ultrasonic beam emitted by said capsule;
reflecting a portion of said ultrasonic beam from human tissue;
converting reflected portion of said ultrasonic beam to electronic signal; and
transmitting RF signal indicative of said converted electric signal from said capsule to said external unit.

18. The method of claim 17 wherein step of transmitting RF signal indicative of said converted electric signal comprises transmitting signal at frequency equal to frequency of said RF power.

19. The method of claim 17 wherein step of transmitting RF signal indicative of said converted electric signal comprises transmitting signal at frequency not equal to frequency of said RF power.

20. The method of claim 17 and further comprising detecting position and orientation of said capsule in respect to said external unit.

21. The method of claim 20 and further comprising calculating a 3D image of boundaries of a body lumen through which said capsule is traversing based on signal indicative of said converted electric signal and said detected position and orientation of said capsule in respect to said external unit.

22. The method of claim 21 and further comprising displaying said calculated 3D image.

23. The method of claim 21 wherein said body lumen through which said capsule is traversing comprises at least a part of a patient's gastrointestinal tract.
